(19) United States
(12) Patent Application Publication
(10) Pub. No.: US 2010/0049163 A1
(43) Pub. Date: Feb. 25, 2010

(54) Drug delivery device, kit and method for peroperative local pharmacological treatment of bone surfaces

(75) Inventor: Per V. Aspenberg, Linkoping (SE)

Correspondence Address: BACON & THOMAS, PLLC, 625 SLATERS LANE, FOURTH FLOOR, ALEXANDRIA, VA, 22314-1176, US

(73) Assignee: Addbio AB, Linköping (SE)

(21) Appl. No.: 12/308,887

(22) PCT Filed: Jun. 20, 2007
(86) PCT No.: PCT/SE2007/000599

§ 371 Date: Sep. 10, 2009

Related U.S. Application Data
(60) Provisional application No. 60/816,744, filed on Jun. 27, 2006.

(51) Int. Cl. A61M 37/00

(52) U.S. Cl. 604/502; 604/57

(57) ABSTRACT

A drug delivery device for local administration of a solution or suspension of a drug, such as a bone modulating drug, to a bone surface, comprising a ductile, absorbent material comprising said drug and being of a shape adapted or adaptable to the bone surface. The drug delivery device may additionally comprise a support of a non-absorbent material and a handle piece. The bone modulating drug may be chosen from bisphosphonates, statins, growth factors, signaling proteins and bone morphogenic protein. The device may be used in peroperatively treating a bone surface by local administration to the surface of the drug.

The present invention relates to the field of orthopaedic surgery and provides a drug delivery device, a kit and a method for peroperative local pharmacological treatment of bone surfaces for effective local administration of particularly a bone modulating drug, such as a bisphophonate, to the bone surface before it is connected to a surgical implant/prosthesis or another bone surface.

### BACKGROUND

It has recently been clinically demonstrated that local treatment of bone with bisphosphonate, the presently most used bone modulating drug, can improve the fixation of a total joint prosthesis in the knee. Many other drugs might become useful in similar ways in several types of joint replacement. The goal is to influence the body's reaction to the implant, by reducing bone resorption, reducing inflammation, stimulating bone formation or in other ways. Most drugs that might become utilized must be applied at high concentrations, with the obvious risk that systemic treatment, e.g. via tablets or injection, will cause unwanted or deleterious side effects on the rest of the body. Therefore local treatment is preferable or necessary for an effective result. With an increasing number of elderly people who are in need of orthopaedic surgery, this is a field of increasing importance where it is of considerable economic importance to avoid the need for re-surgery.

Drugs for improving implant fixation in humans, in particular bishosphonates, have so far been applied by dripping a solution onto a somewhat dry, porous bone surface, which can accommodate the applied fluid volume. This requires that the surface is easily accessible within the surgical wound, as dry as possible (i.e. not bleeding) and reasonably horizontal.

For stimulation of fracture repair, drugs have been applied within the haematoma surrounding the fracture, usually together with a carrier with slow-release properties. For improving implant fixation, this is usually not possible, because once the implant is in place; it mechanically blocks access for the drug to the bone surface.

Problems Encountered with Current Techniques
a) The bone is often bleeding, so that an applied solution is flushed away.
b) The surface to be treated may be essentially vertical, so that a fluid cannot be effectively dripped or poured onto it.
c) The solution may be spilt in many areas of the wound, so that it is quickly resorbed and enters general circulation in uncontrolled amounts.
d) The part of the solution that is not absorbed by the bone will pour out in the wound and add to the total body dose for no good.

For example, in total hip replacements a cup is inserted in the pelvic bone. It would be desirable to locally treat the bone surface with drugs facilitating bone modulation before insertion of the cup. As a cup-shaped cavity is surgically created in the pelvis, the bone bleeds, the cavity is tilting more than 45 degrees and soft tissues reduce access. If a solution is poured into the cavity, it will only treat the bottom and hardly penetrate the bone, because the bleeding goes in the other direction. Therefore, it will be impossible to even estimate how large a proportion of the drug gets into contact with the bone (in case of bisphosphonates contact is enough, because they bind effectively to the bone surface).

Another example of an indication for local treatment with a bisphosphonate is the kind of hip prostheses where the head of the femur is preserved and covered with metal. In this case the head is machined to a cylindrical shape before the metal is put on. This cylinder should be treated around its circumference, which is hardly accessible.

### DESCRIPTION OF THE INVENTION

The present invention provides in one aspect a drug delivery device for local administration of a solution or suspension of a drug to a bone surface, comprising a ductile, absorbent material comprising said drug and being of a shape adapted or adaptable to the bone surface.

An embodiment of the invention provides a support, e.g. in the form of a plate, to the drug delivery device of a non-absorbent material, such as wood, plastic or metal material.

According to another embodiment of the invention, the drug delivery device is associated with a rod, for easier application of the drug delivery device. In an example, the rod may have a flat lower surface, or a surface with a curvature, for easier contact with a bone surface. In another example, the rod has a central longitudinal cavity for administration of the solution or suspension of the drug. To facilitate ease of operation, the device may additionally have a handle piece in association with the device of the invention.

In a further embodiment of the invention, the drug delivery device is pre-treated with a bone modulating drug. Examples of such bone modulating drugs include bisphosphonates, such as alendronate, zoledronate, ibandronate and pamidronate; statins; growth factors; signalling proteins; and bone morphogenic protein.

In another aspect of the invention there is provided a kit containing, in one or several aseptic or sterile package(s), a disposable drug delivery device according to the invention.

Yet another aspect of the invention is directed to a method of peroperatively treating a bone surface by local administration to the surface of a drug with a drug delivery device according to the invention.

### SHORT DESCRIPTION OF THE DRAWINGS

FIG. 1 shows a drug delivery device in use, in association with a handle piece.

FIGS. 2-5 show the drug delivery device with a shape commensurate with the respective implant shape.

FIG. 6 shows a drug delivery device that allows supplementary administration of drugs, in addition to the initial bone modulating drug.

### DETAILED DESCRIPTION OF EMBODIMENTS

The invention shall now be described, with special reference to the accompanying figures.

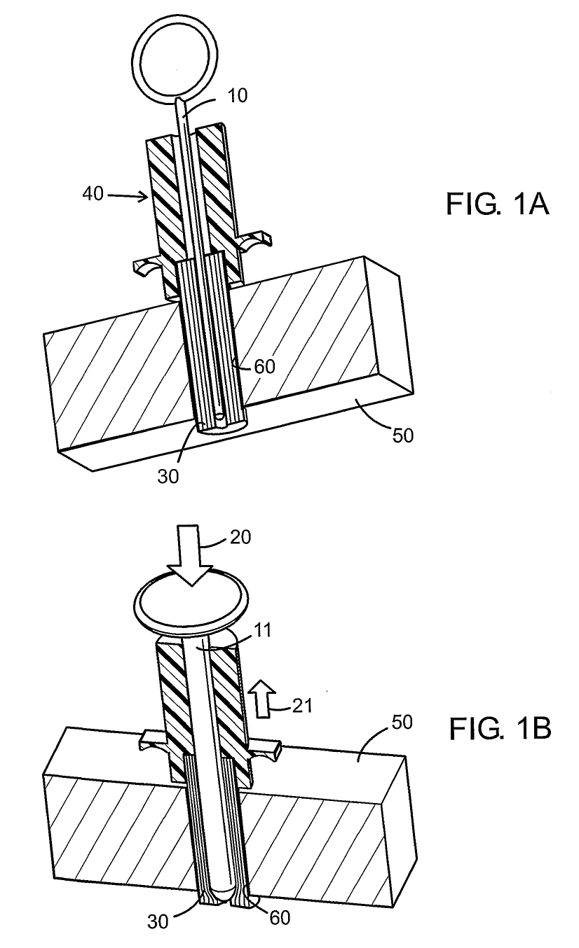

In the specific embodiments described below, the drug delivery device loaded with a drug solution or suspension is shown as the hatched part of the figures. In FIG. 1 bore holes 60 appear in the bone 50. An embodiment or the invention is illustrated, in FIGS. 1a and 1b, wherein FIG. 1a shows a rod 10 pushing the drug delivery device 30 into position. FIG. 1b shows how another rod 11 with a greater diameter is pushed in the direction 20 and used to squeeze the drug solution or suspension into contact with the bone surface, whereupon it is left in place for a sufficient time period and finally lifted in the direction 21. The device is associated with separate handle pieces 40, facilitating one-hand operation of the device.

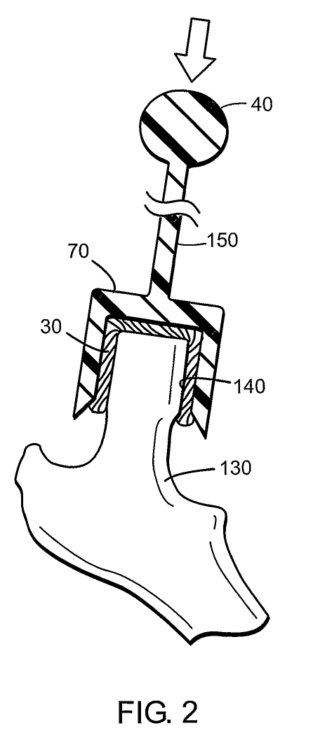

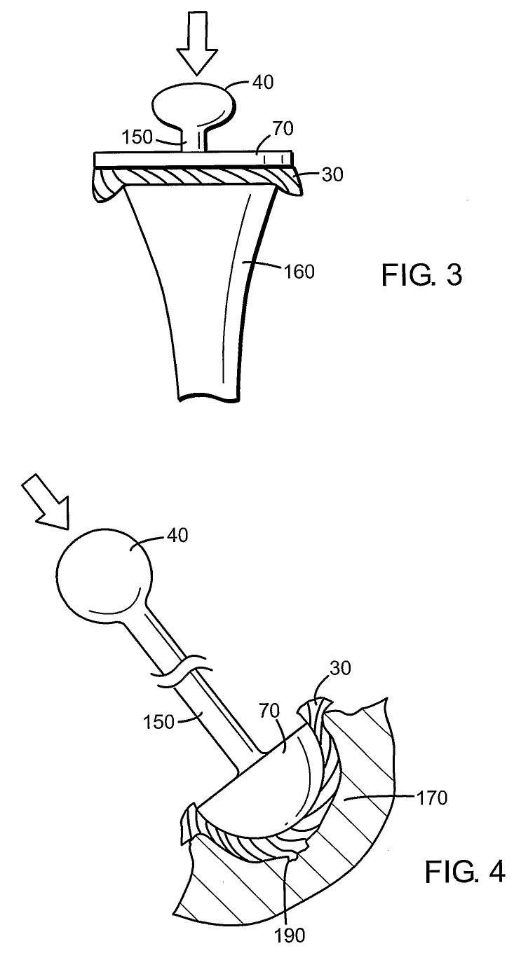

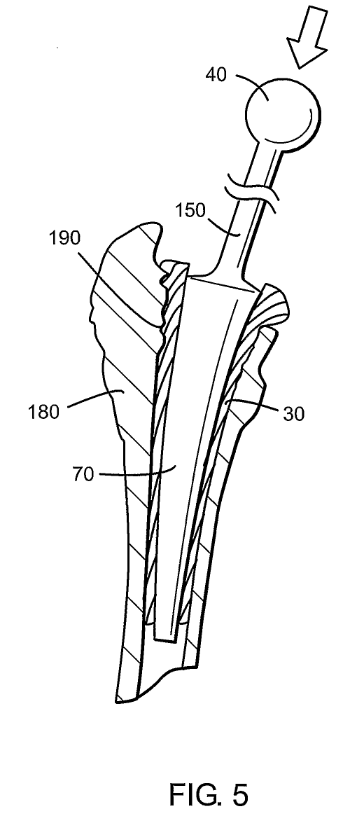

FIGS. 2-4 show a few examples of placing the drug delivery device against a bone surface prepared for association with an implant, whereby the drug delivery device 30 is provided with a handle piece 40. An extension 150 of the handle piece 40 ensures that the handle piece is located at a suitable distance from the bone during use. The drug delivery device has a shape commensurate with the respective implant shape, provided by the metal support plate 70. FIG. 2 shows the drug delivery device being located against the proximal femur 130, whereby the drug delivery device 30 is closely pressed against the cut and shaped bone surface 140. FIGS. 3, 4 and 5 show the drug delivery device against the proximal tibia 160, the pelvic acetabulum 170 and the proximal femur 180, respectively. In all of these figures, the drug delivery device 30 is via a metal support 70 via an extension 150 connected to a handle piece 40. FIGS. 4 and 5 in particular show how irregularities 190 in the bone are filled up by the drug delivery device 30.

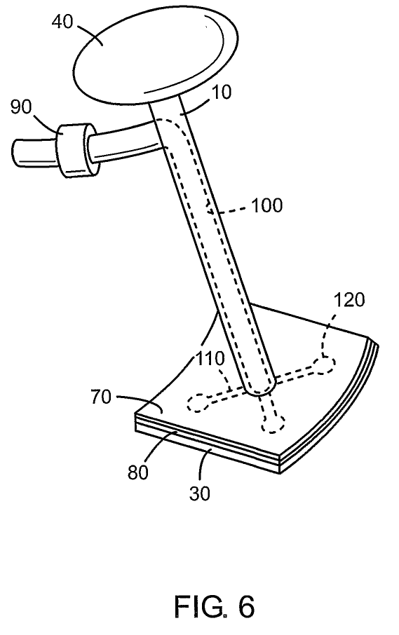

FIG. 6 shows an embodiment of the drug delivery device with a handle piece 40 connected to a rod 10, wherein a central longitudinal cavity 100 runs towards a metal support plate 70, and via connecting tubing 110 ends in apertures 120 therethrough. Below the rod the metal support plate 70 is located, which is connected to the backside of the drug delivery device 30, in turn optionally fitted with a polymeric coating 80. The upper opening of the cavity in the rod is fitted with a valve 90.

The drug delivery device is mainly adapted or adaptable to fit to the bone surface where an implant or similar device will subsequently be applied, so that a suitable amount of the drug can easily be applied to the bone surface in a pre-treatment step before e.g. attachment of the implant or similar device. Alternatively, the drug delivery device may be used for the pre-treatment of orthopaedic bore holes in bone or fractures. In order to fit to the bone surface the drug delivery device should have substantially the same form as the implant, or be made of a ductile material.

The term implant-like surface or form is henceforth used to describe that the form of the drug delivery device contacting the bone surface should for certain applications be similar to the bone-contacting surface of the implant that will be applied subsequently. The material of the drug delivery device is chosen so that it easily absorbs and carries the drug, e.g. after dipping the drug delivery device in a drug solution or suspension. Non-exclusive examples of materials for manufacture of the drug delivery device are presented below. These examples include absorbing haemostatic products, such as gelatine sponges (for example Spongostan™) and other synthetic and natural sponges (such as sea sponge), alternatively woven or non-woven paper products. Moreover, a natural or synthetic textile material, such as gauze or a non-woven textile material, such as cotton wool, may be used, for example surgical cloths and surgical pads. These materials must alone or in combination have adequate absorbing characteristics, i.e. need to have a suitable thickness for drugs in solution or suspension to leave the material in suitable quantities when the material is pressed against a bone surface. Moreover, the material needs to allow easy sterilization using standard techniques (i.e. irradiation, treatment with ethylene oxide and in particular steam treatment in an autoclave).

The drug delivery device needs to be adapted or adaptable to the bone surface, when it is applied to the bone surface (having or receiving an implant-like form) and this can be achieved in several ways. The drug delivery device, as such, can be given the right form during manufacture, for a specific application, and may be provided with a somewhat stiffer backside which helps establishing a substantially equal pressure when the device is pressed against the bone surface, for substantially even distribution of the drug solution or suspension over the bone surface. Alternatively, the drug delivery device is made out of a ductile material, obtaining the shape of a support structure or being shaped manually. According to such an alternative embodiment of the invention a support structure, e.g. a plate, is provided with an implant-like surface. For example a sheet of the drug delivery device material may be fixed to the support structure and formed accordingly. The support structure, which may be impermeable or semi-permeable, is in one embodiment made out of a polymeric material. The polymeric material may be in the form of an adhesive film mounted on the device. In another embodiment, the support is a thermoplastic film heat mounted on the device. The support structure may be arbitrarily selected from films according to the state of the art. More than one support structure may be used, for example a polymeric backing in combination with a metal plate (see FIG. 6). The polymeric backing may then self-adhere to the support plate. In order to access a deeply located bone surface a rod may be used to press the drug delivery device, preferably carried by a support structure, against the bone surface. The rod being fixed to the support plate is here a convenient part of the device. As there are a fairly limited number of implant designs for a certain type of surgery, a reasonably small series of devices with individual support conformations will cover the need. The rod is in one embodiment provided with a central, longitudinal cavity, for administration of the bone-modulating drug or administration of other drugs that may be beneficial to administer, especially when the device should not be removed due to possible, unwanted bleeding. A further embodiment of a rod with a longitudinal cavity is fitted with a valve, and a pump for easy and controllable administration of drugs. In still another embodiment, such a rod is fitted with a compressed air valve, for pressurized administration of drugs. The rods are either disposable, or multi-use items. Disposable rods may be manufactured from a polymeric material, whereas rods for multiple use are manufactured from state of the art metal material for surgical instruments.

The drug delivery device is provided as a sterile and physiologically compatible material with the ability to absorb a sufficient amount of solution or suspension, e.g. around 1 ml for knee joint surgery. The thickness of the drug delivery device is in the above example around 0.5 cm. It should be thin enough to be easy to handle and put in place and thick enough to carry the desired amount of liquid. A push via the rod should be sufficient to press out the liquid over the bone surface.

The drug delivery device may be fixed to the support/plate surface in many different ways, e.g. by a glue or an adhesive surface structure of a type known from many areas. Even a clamping device can be used.

In one embodiment, a device comprising a handle piece is used to initially put the drug delivery device in place, and thereupon guide the rod. This handle piece device finds its application primarily for bore holes in bone (see FIG. 1).

In the method for treatment the drug delivery device is pushed against the bone, so that the drug solution is squeezed out of it and is pressed into the porous bone matrix. Of importance is that according to this method a slight pressure is created that stops bleeding and allows the solution to enter the bone pores. Using the rod described above gives a more uniform dispensing of the drug(s). When the pressure on the plate is released, superfluous solution is reabsorbed by the drug delivery device. A drug, which has affinity for the bone tissues (such as a bisphosphonate) will remain in the bone due to the interaction with the bone matrix. The drug delivery device as such, optionally equipped with a somewhat stiffer back side, or a supporting plate is shaped to substantially fit the bone surface to be treated (an implant-like form). For example, in the case of the above mentioned cavity in the pelvis (see FIG. 4), the plate could be shaped as the cup that will later be implanted. Most orthopaedic implants have, as mentioned above, standard shapes (see e.g. FIGS. 2, 3 and 5, showing the proximal femur, proximal tibia and proximal femur, respectively). The invented device, a drug delivery device with an implant-like surface form, optionally placed on a support structure, and comprising a suitable dose of an appropriate drug, could be delivered by the manufacturer together with each implant. Because the device has a shape that closely fits the bone surface, a rather small fluid volume can be used and leakage outside the surface area to be treated is minimized. It is also possible to have a sealing or barrier at the periphery of the plate constituting a part of the plate, such as a rubber ring, which is an optional detail comprised by embodiments of the invention.

Drugs to be administered in accordance with the present invention are preferably bone modulating substances with bishosphonates like alendronate or zoledronate, as the presently most important examples. The drug can be delivered in a separate ampoule and being applied to the drug delivery device just before use or the drug delivery device can be ready for use as delivered. The method and the device can also be used for other types of drugs which are found to be of importance in connection with surgery.

The device can be provided ready for use after removal of packaging material keeping it sterile or aseptic prior to use. It can also be provided with the drug solution in a separate container whereby the solution is transferred to the drug delivery device just before use. It is of course also possible to prepare a set of support bodies (plates) equipped with rods which are sterilized and reused and only require a drug delivery device to be fixed to the implant-like surface. The exchange part is here the drug delivery device which is prepared as described above and adopted to the plate before use. The devices may be packed individually, or in packages with multiple devices. In one embodiment, the drug delivery device is delivered on a reel in a sterile package, whereby it is possible to cut off the suitable length of the device. An embodiment of the invention provides an aseptic or sterile package of at least one drug delivery device together with at least one rod. The above packages may provide protection by way of a protective foil.

A further embodiment of the invention is a method for local administration of a bone modulating substance to a bone surface using a device as described above. The device with the drug delivery device soaked with the substance is pressed against the bone surface whereby the substance is squeezed out of the drug delivery device and a volume thereof is absorbed by the bone matrix. When the pressure is released excess substance solution is reabsorbed.

According to still a further embodiment of the invention a method is provided where a drug is applied to a bore hole in bone, e.g. in orthopaedic fracture surgery and for dental implants (see FIG. 1). These holes are usually filled with blood. The principle for application of the drug is again that a drug delivery device with an appropriate form (e.g. a cylinder) is squeezed between the bone and a supporting structure which is the same as described above. In this case a thin removable central rod may be used to position the tube shaped drug delivery device in the bore hole. The drug delivery device has a dimension suitable to cover the inner surface of the hole. After insertion of the drug delivery device in the hole, the rod is pulled out and replaced by a thicker rod, preferably with a conical tip, so that the drug delivery device is squeezed against the walls of the bore hole. Possibly a thin gliding layer is required between the drug delivery device material and the rod. This can easily be achieved by providing the back of the drug delivery device surface (the one not in contact with the bone surface) with a silicone layer or a similar low friction material, e.g in a tube form. The end of the drug delivery device that does not enter the bore hole can be equipped with a handle piece preferably attached to the low friction surface layer so that the push upon the expander pin is balanced by a pulling force via this handle, making it possible to perform the whole manoeuvre with one hand.

Even though the invention has been described particularly with respect to the examples and figures, it should be understood that these are only made to illustrate the essential features of the invention, and that deviations from the disclosed details may naturally be made by a man skilled in the art while still being comprised by the invention as claimed.

1. A drug delivery device for local administration of a solution or suspension of a drug to a bone surface, comprising a ductile, absorbent material comprising said drug and being of a shape adapted or adaptable to the bone surface.

2. The drug delivery device according to claim 1, wherein the absorbent material has a support of a non-absorbent material.

3. The drug delivery device according to claim 1, wherein the absorbent material and/or support is in association with a rod that has at its one end a surface which is adapted or adaptable to the bone surface.

4. The drug delivery device according to claim 3, wherein the rod surface is flat or has a curvature.

5. The drug delivery device according to claim 3, wherein the rod has a central longitudinal cavity for administration of the solution or suspension of the drug.

6. The drug delivery device according to claim 1, wherein the device additionally has a handle piece.

7. The drug delivery device according to claim 1, wherein the drug is a bone modulating drug.

8. The drug delivery device according to claim 7, wherein the bone modulating drug is chosen from the group consisting of bisphosphonates, statins, growth factors, signaling proteins and bone morphogenic protein.

9. A kit containing in one or several aseptic or sterile package(s) a disposable drug delivery device according to claim 1.

10. A method of peroperatively treating a bone surface by local administration to the surface of a drug with a drug delivery device according to claim 1.

11. The drug delivery device according to claim 2, wherein the absorbent material and/or support is in association with a rod that has at its one end a surface which is adapted or adaptable to the bone surface.

12. The drug delivery device according to claim 4, wherein the rod has a central longitudinal cavity for administration of the solution or suspension of the drug.

13. The drug delivery device according to claim 2, wherein the device additionally has a handle piece.

14. A kit containing in one or several aseptic or sterile package(s) a disposable drug delivery device according to claim 2.

15. A method of peroperatively treating a bone surface by local administration to the surface of a drug with a drug delivery device according to claim 2.
